(19) United States
(12) Patent Application Publication
(10) Pub. No.: US 2005/0196430 A1
(43) Pub. Date: Sep. 8, 2005

(54) Antimicrobial enamel glaze

(75) Inventors: Anders Olsson, Satofta (SE); Howard Wayne Swofford, Newton, NC (US)

Correspondence Address: KENNEDY COVINGTON LOBDELL & HICKMAN, LLP, 214 N. TRYON STREET, HEARST TOWER, 47TH FLOOR, CHARLOTTE, NC, 28202, US

(73) Assignee: MICROBAN PRODUCTS COMPANY, Huntersville, NC (US)

(21) Appl. No.: 11/067,881

(22) Filed: Feb. 28, 2005

Related U.S. Application Data
(63) Continuation-in-part of application No. 11/040,379, filed on Jan. 21, 2005.
(60) Provisional application No. 60/538,074, filed on Jan. 21, 2004.
(60) Provisional application No. 60/567,671, filed on May 3, 2004.

(51) Int. Cl. C03C 3/14; A61K 33/32

(52) U.S. Cl. 424/443; 424/641; 501/49

(57) ABSTRACT

An antimicrobial enamel glazing composition comprising zinc borate for imparting antimicrobial characteristics to numerous enameled products. A method for producing the antimicrobial glazing composition and coating an article with the the antimicrobial enamel glazing composition.

### CROSS-REFERENCE TO RELATED APPLICATIONS

This application is entitled to the benefit of and claims priority to U.S. patent application Ser. No. 11/040,379, filed on Jan. 21, 2005, and to provisional U.S. Patent Application Ser. No. 60/538,074, filed on Jan. 21, 2004, and provisional U.S. Patent Application Ser. No. 60/567,671, filed on May 3, 2004, each of which is incorporated herein by reference in its entirety.

### FIELD OF THE INVENTION

The present invention relates to the field of antimicrobial protection. More specifically, the present invention relates to a composition for imparting built-in and long lasting antimicrobial characteristics to ceramic and metal products. In particular, the present invention pertains to glazing compositions that impart built-in antimicrobial characteristics to ceramic and metal products.

### BACKGROUND OF THE INVENTION

The field of providing products with built-in antimicrobial protection has grown tremendously over the past several years. What once started out as a premium or novel option for high-end consumer products and medical devices has now grown into a mainstream characteristic found in many consumer products. Consumers can go to any home improvement center and see dozens if not hundreds of products that claim some degree of resistance to microbiological growth or contamination. Some major retailers have specific sections devoted to such antimicrobial products.

Antibacterial products have been introduced into plastics, textiles, liquids, metal coatings and an array of other types of materials. However, there remain several areas of consumer and commercial products in which development of commercially viable antimicrobial products has proven difficult. One such area is ceramic and enamel coatings.

Ceramic coatings are commonly used in products that store, treat, or transport water and liquid waste. Ceramic toilets, urinals, bidets, bathroom basins, flooring tiles and other bathroom fixtures are probably the most common example of such products.

Ceramic products used to collect and transport water are often stained by scum and films of biologic origin (e.g., bacteria, fungus, mold, mildew). To date, the primary method of removing biological scum and film from these ceramic products has been to abrade the ceramic surface in the presence of topical cleaning agent. This process is time consuming and provides little or no protection against future growth. Some cleaning agents can damage the surface of the ceramic product. Therefore there is great interest in the development of ceramic coatings that have built-in protection against the growth and proliferation of microbes.

A few such built-in antimicrobial coatings are described in the ceramic literature but they have not seen commercial success. Existing technologies are somewhat limited. For example, the high temperatures used in ceramic firing processes typically preclude the use of organic antimicrobial agents. Inorganic silver-based antibacterials are too expensive. Zinc oxide is known as having antimicrobial characteristics and has been used in the preparation of ceramic glazing compositions. However, known ceramic glazing compositions that rely solely upon zinc oxide as an antimicrobial agent have not shown antimicrobial efficacy sufficient for control of microbial growth and proliferation on ceramic surfaces. Accordingly, there is a need for a low-cost ceramic or enamel coating that has built-in antimicrobial protection.

Thus, one object of the present invention is to provide a new and useful antimicrobial ceramic coating that can impart antimicrobial characteristics in a wide range of products.

A still further object of the invention is to provide this ceramic coating at a cost that is acceptable to the marketplace. Furthermore, this antimicrobial ceramic coating should be safe to humans, exhibit commercially acceptable antimicrobial properties, and most importantly, be compatible with existing ceramic production processes.

These and other objects are achieved by the present invention, which in one embodiment is an antimicrobial ceramic glazing composition comprising a quantity of zinc borate sufficient to achieve a commercially acceptable level of antimicrobial efficacy. In preferred embodiments this ceramic glazing composition also comprises a quantity of zinc oxide.

In a further embodiment, the invention is a ceramic article that exhibits antimicrobial properties. The ceramic article according to the invention has at least one surface and a glaze on a portion of that surface. The glaze comprises a quantity of zinc borate sufficient to achieve a commercially acceptable level of antimicrobial efficacy. In preferred embodiments this ceramic glazing composition also comprises a quantity of zinc oxide.

In yet another embodiment, the invention is a method of making an antimicrobial ceramic glaze and a method of making a ceramic article comprising the antimicrobial ceramic glaze.

Another object of the present invention is to provide a new and useful antimicrobial enamel glaze that can impart antimicrobial characteristics in a wide range of products.

Still yet a further object of the invention is to provide this enamel glaze at a cost that is acceptable to the marketplace. Furthermore, this antimicrobial enamel glaze should be safe to humans, exhibit commercially acceptable antimicrobial properties, and most importantly, be compatible with existing enamel production processes.

These and other objects are achieved by the invention, which in one embodiment is an antimicrobial enamel glazing composition comprising a quantity of zinc borate sufficient to achieve a commercially acceptable level of antimicrobial efficacy. In preferred embodiments this enamel glaze composition also comprises a quantity of zinc oxide.

In a further embodiment, the invention is an enameled article that exhibits antimicrobial properties. The enameled article according to the invention has at least one surface and a glaze on a portion of that surface. The glaze comprises a quantity of zinc borate sufficient to achieve a commercially acceptable level of antimicrobial efficacy. In preferred embodiments this enamel glaze composition also comprises a quantity of zinc oxide.

### DETAILED DESCRIPTION

As used herein, the terms “microbe” or “microbial” should be interpreted to encompass any of the microscopic organisms commonly studied by microbiologists. Such organisms include, but are not limited to, bacteria and fungi as well as other single-celled organisms such as mold, mildew and algae. Viral particles and other infectious agents are also included in the term microbe.

For ease of discussion, this detailed description may make reference to bacteria and antibacterial agents. This method of presentation should not be interpreted as limiting the scope of the invention in any way.

One embodiment of the present invention relates to ceramic coatings and in particular to ceramic glazing on the outer surfaces of ceramic products. The following brief discussion of vitreous china or ceramic production is provided as an aid to the reader. This discussion is presented in the context of the production of bathroom fixtures. Those skilled in the art recognize that the production process of other ceramic products may vary from that which is presented below. The claimed invention, however, is adaptable to any such variances.

The initial stage of a typical ceramic production process is the production of barbotine or slip, a clay from which bathroom ceramic products are made. Barbotine is made from a mixture of clays, kaolin, phyllites, feldspar and quartz.

Individual pieces are cast by pouring the barbotine into molds made of gypsum or microporous resin.

In the casting processes that use gypsum molds the parts are formed by absorption of water contained in the barbotine through the capillary action of the gypsum. As water leaves the barbotine the part solidifies to a point where the mold can be opened. The still malleable part is then removed from the mold.

Casting processes that use resin molds are called “high pressure” processes. Parts are formed by filtering water contained in the barbotine clay through micropores in the resin molds by the application of pressure. The water is eliminated by injecting compressed air along the molds.

Generally, gypsum molds are used for making parts with a more complex geometry and that are produced in low volumes. Resin molds are used for parts whose geometry is simpler and that are produced in high volumes.

After casting and removal from the molds, the parts go for drying in kilns under controlled humidity and temperature (approximately 90° C.). The drying cycle lasts about 7 hours, reducing the water content of the part from about 16% to less than 1%. Following this, the parts are inspected to detect possible flaws. The parts then go to the coating process. The coating process is often referred to as the glazing step.

The glazing step typically comprises the manual application of ceramic glaze on the parts using guns in individual booths fitted with exhaust systems and water curtains. Typical ceramic glaze is produced from a mixture of kaolin, feldspar, quartz, colorings and other additives. Once coated, the parts are fired in continuous kilns, reaching temperatures of about 1,250° C., in an approximately 15-hour cycle. The firing process gives the glazed part the color and transparent appearance that is typical of vitreous china.

In one embodiment, the invention is a ceramic glazing composition that provides commercially acceptable antimicrobial efficacy after the firing process. In other words, the claimed glazing reduces or substantially eliminates the growth and proliferation of microbes on the surface of ceramic articles upon removal from the firing process and without any further treatment (e.g., further coating or painting of the ceramic part).

The antimicrobial ceramic glazing composition according to the invention comprises components commonly utilized in the preparation of ceramic glazing plus a quantity of zinc borate sufficient to achieve a commercially acceptable level of antimicrobial efficacy.

Zinc borate is the common term for a hydrated mineral-like substance. Zinc Borate is most often used as a flame retardant and smoke suppressant additive but it is sometimes used as an antifungal agent. However, zinc borate is not known as an antimicrobial agent in ceramic coatings.

The quantity of zinc borate required to achieve a commercially acceptable level of antimicrobial efficacy for finished ceramic products may vary depending upon the level of contamination generally associated with the product. However, the concentration of zinc borate for most commercial applications is at least about 5,000 ppm of zinc borate. Concentrations above about 100,000 ppm can have adverse impacts on the aesthetic qualities of the ceramic glaze. Concentrations of about 20,000 ppm to about 40,000 of zinc borate are preferred.

In an alternative embodiment, the glazing composition according to the invention also comprises a quantity of free zinc oxide. Free zinc oxide, as used herein, refers to an additional amount of zinc oxide that is added to the glazing composition separate from the zinc borate.

In the embodiments that employ a combination of zinc borate and zinc oxide, the ratio of zinc borate to zinc oxide in the glazing composition may range from about 90:10 to about 10:90. A ratio of about 50:50 is preferred but may be altered depending upon cost considerations. Preferably, a ratio of about 50:50 of zinc borate to zinc oxide is added to the ceramic glazing composition at about 2 weight % for the combination based upon the weight of the ceramic glaze composition.

Similar to the embodiment that employs zinc borate, ceramic glazing compositions comprising zinc borate and zinc oxide may have at least about 5,000 ppm of a combination of zinc borate and zinc oxide. Preferably, ceramic glazing compositions comprising a combination of zinc borate and zinc oxide have about 20,000 ppm to about 40,000 ppm of the combination. More preferably, about 20,000 ppm of the combination. Such concentrations, in varying ratios, achieve at least a 90% reduction, preferably greater than a 99% reduction, of microbial species as compared to an untreated control applied to a ceramic surface coated with the glazing composition. Concentrations above 100,000 ppm may have adverse aesthetic effects on the glazing.

If desired, other antimicrobial agents capable of surviving the high temperatures of the glazing process may be added to the glazing composition. Such agents include, but are not limited to, silver (e.g., silver salts and silver zeolites), copper, and other known metallic antimicrobial agents. Such agents can be added in relatively minor amounts to supplement biocidal activity against specific pathogens. Of course, such metallic antimicrobial agents can be added in greater quantities if desired.

In a further embodiment, the invention encompasses a ceramic article that exhibits antimicrobial properties. The claimed antimicrobial ceramic article comprises a ceramic substrate having at least one surface and a glaze on at least a portion of that surface. The glaze utilized in this embodiment of the invention is the same as that described in the first embodiment of the invention. In other words, the glaze comprises a quantity of zinc borate or a combination of zinc borate and zinc oxide that is sufficient to achieve a commercially acceptable level of antimicrobial efficacy.

In a preferred embodiment, the glazing will comprise at least about 5,000 ppm of zinc borate. Concentrations above about 100,000 ppm of zinc borate may have adverse aesthetic effects on the glazing. Concentrations of about 20,000 ppm to about 40,000 ppm of zinc borate are preferred. Alternatively, the glaze comprises a combination of zinc borate and zinc oxide, where the combined concentration of zinc borate and zinc oxide is preferably at least about 5,000 ppm. Preferably, the combined concentration of zinc borate and zinc oxide is in a range of about 20,000 ppm to about 40,000 ppm. If a combination of zinc oxide and zinc borate is used, the ratio of zinc borate to zinc oxide may be about 90:10 to about 10:90, preferably about 50:50.

In a still further embodiment, the invention encompasses a method of making an antimicrobial ceramic glaze and an article having an antimicrobial ceramic glaze.

The antimicrobial ceramic glaze according to the invention may be made by adding zinc borate or a combination of zinc borate and zinc oxide, both of which are commercially available from a number of sources, to an existing glazing composition. Those skilled in the art of preparing glazing compositions will recognize that the zinc borate and zinc oxide may be added separately or in combination at any point in the process of making the glazing composition.

Of course, care should be taken to ensure that the quantity of zinc borate (or a combination of zinc borate and zinc oxide) is sufficient to achieve a commercially acceptable level of antimicrobial efficacy. In preferred embodiments the quantities of these antimicrobial agents are the same as those set forth above in the discussion of the glazing composition.

The method of making the claimed antimicrobial ceramic article closely resembles the general method for making ceramic articles set forth at the beginning of the detailed description. However, in the method according to the invention, upon removal of the article from the mold, the article is coated with the antimicrobial ceramic glazing composition according to the invention. The coated article is then fired as usual with the ceramic coating retaining its antimicrobial characteristics even after the firing.

As noted previously, the ceramic glaze according to the invention was designed to impart built-in antimicrobial protection to a variety of ceramic articles. Accordingly, the scope of the invention includes ceramic articles that incorporate the glazing according to the invention. Such articles include, but are not limited to, toilets, bidets, washbasins, towel rails, soap holders, toilet roll holders, water control fixtures (e.g., hot and cold water handles), ceramic tiles, and other ceramic applications.

Another embodiment of the present invention relates to enamel coatings and in particular to enamel glaze on the outer surfaces of metal products. Enamel is a form of low temperature ceramic glaze that is usually applied over metal. This provides metal products with the resistance to wear and corrosion of ceramics while having the physical strength and durability of metal. Typical firing temperatures for enamel glaze are approximately 600 to 1000° C. as compared with firing temperatures of approximately 1200 to 1300° C. for ceramic glaze. Typical products that may be enameled are kitchen and bathroom sinks, bathtubs, eating utensils, pots and pans, jewelry, and decorative items, among others.

Before applying enamel, the product to be enameled, usually metal, is molded or formed into the shape that is desired for the application. For example, a sink will be drawn down from a sheet of metal into its final shape before applying the enamel glaze. The enamel glazing components are mixed and milled to the desired particle size in water to form a slurry, and then the slurry is sprayed or otherwise coated onto the metal, dried, and then the part is fired in a kiln at approximately 800° C. The glaze imparts color, luster, and the hardness and chemical resistance of ceramic to the metal part. Those skilled in the art recognize that the production process of other enameled products may vary. The present invention, however, is adaptable to any such variances.

The coating process is often referred to as the glazing step. The glazing step typically comprises the manual application of glaze on the articles or parts. Typical enamel glaze may be produced from a mixture of kaolin, feldspar, quartz, colorings and other additives.

In one embodiment, the invention is an enamel glazing composition that provides commercially acceptable antimicrobial efficacy after the firing process. In other words, the enamel glazing reduces or substantially eliminates the growth and proliferation of microbes on the surface of metal articles upon removal from the firing process and without any further treatment (e.g., further coating or painting of the metal part).

The antimicrobial enamel glazing composition according to the invention comprises components commonly utilized in the preparation of enamel glazing plus a quantity of zinc borate sufficient to achieve a commercially acceptable level of antimicrobial efficacy.

Zinc borate is the common term for a hydrated mineral-like substance. Zinc Borate is most often used as a flame retardant and smoke suppressant additive but it is sometimes used as an antifungal agent. However, zinc borate is not known as an antimicrobial agent in ceramic coatings.

The quantity of zinc borate required to achieve a commercially acceptable level of antimicrobial efficacy for finished enamel products may vary depending upon the level of contamination generally associated with the product. However, the concentration of zinc borate for most commercial applications is at least about 5,000 ppm of zinc borate. Concentrations above about 100,000 ppm can have adverse impacts on the aesthetic qualities of the ceramic glaze. Concentrations of about 20,000 ppm to about 40,000 of zinc borate are preferred.

In an alternative embodiment, the enamel glazing composition according to the invention also comprises a quantity of free zinc oxide. Free zinc oxide, as used herein, refers to an additional amount of zinc oxide that is added to the enamel glazing composition separate from the zinc borate.

In the embodiments that employ a combination of zinc borate and zinc oxide, the ratio of zinc borate to zinc oxide in the enamel glazing composition may range from about 90:10 to about 10:90. A ratio of about 50:50 is preferred but may be altered depending upon cost considerations. Preferably, a ratio of about 50:50 of zinc borate to zinc oxide is added to the enamel glazing composition at about 2 weight % for the combination based upon the weight of the enamel glaze composition.

Similar to the embodiment that employs zinc borate, enamel glazing compositions comprising zinc borate and zinc oxide may have at least about 5,000 ppm of a combination of zinc borate and zinc oxide. Preferably, ceramic glazing compositions comprising a combination of zinc borate and zinc oxide have about 20,000 ppm to about 40,000 ppm of the combination. More preferably, about 20,000 ppm of the combination. Such concentrations, in varying ratios, achieve at least a 90% reduction, preferably greater than a 99% reduction, of microbial species as compared to an untreated control applied to a metal surface coated with the enamel glazing composition. Concentrations above 100,000 ppm may have adverse aesthetic effects on the glazing.

If desired, other antimicrobial agents capable of surviving the temperatures of the enamel glazing process may be added to the enamel glazing composition. Such agents include, but are not limited to, silver (e.g., silver salts and silver zeolites), copper, and other known metallic antimicrobial agents. Such agents can be added in relatively minor amounts to supplement biocidal activity against specific pathogens. Of course, such metallic antimicrobial agents can be added in greater quantities if desired.

In a further embodiment, the invention encompasses an enameled article that exhibits antimicrobial properties. The antimicrobial enameled article comprises a metal substrate having at least one surface and a glaze on at least a portion of that surface. The glaze utilized in this embodiment of the invention is the same as that described previously for enamel glaze. In other words, the enamel glaze comprises a quantity of zinc borate or a combination of zinc borate and zinc oxide that is sufficient to achieve a commercially acceptable level of antimicrobial efficacy.

In a preferred embodiment, the enamel glazing will comprise at least about 5,000 ppm of zinc borate. Concentrations above about 100,000 ppm of zinc borate may have adverse aesthetic effects on the glazing. Concentrations of about 20,000 ppm to about 40,000 ppm of zinc borate are preferred. Alternatively, the enamel glaze comprises a combination of zinc borate and zinc oxide, where the combined concentration of zinc borate and zinc oxide is preferably at least about 5,000 ppm. Preferably, the combined concentration of zinc borate and zinc oxide is in a range of about 20,000 ppm to about 40,000 ppm. If a combination of zinc oxide and zinc borate is used, the ratio of zinc borate to zinc oxide may be about 90:10 to about 10:90, preferably about 50:50.

In a still further embodiment, the invention encompasses a method of making an antimicrobial enamel glaze and an article having an antimicrobial enamel glaze.

The antimicrobial enamel glaze according to the invention may be made by adding zinc borate or a combination of zinc borate and zinc oxide, both of which are commercially available from a number of sources, to an existing glazing composition. Those skilled in the art of preparing glazing compositions will recognize that the zinc borate and zinc oxide may be added separately or in combination at any point in the process of making the glazing composition.

Of course, care should be taken to ensure that the quantity of zinc borate (or a combination of zinc borate and zinc oxide) is sufficient to achieve a commercially acceptable level of antimicrobial efficacy. In preferred embodiments the quantities of these antimicrobial agents are the same as those set forth above in the discussion of the enamel glazing composition.

### EXAMPLES

### Example 1

Several ceramic articles were prepared to test the antimicrobial characteristics of the recited glaze which comprises a combination of zinc borate and zinc oxide. The test articles comprised an underlying ceramic substrate made from a standard commercial barbotine. The glaze used in the testing was a standard glaze comprising silica sand, feldspar, calcium carbonate, china clay, zirconium silicate, a small amount of CMC as a binder, and a small amount of zinc oxide. To this basic glaze composition was added varying quantities of zinc borate and zinc oxide. The glaze composition according to the invention was applied to the articles by spraying. The articles were then fired at 1200° C. One test article was prepared without any additional zinc oxide or zinc borate for use as a control.

Six samples and one control were prepared in accordance with the following table.

TABLE 1

| | Antimicrobial | Ratio of |
|---|---|---|
| Sample | Concentration in the Glaze | Zinc Borate to Zinc Oxide |
| 1 | 20,000 ppm | Zinc Borate:Zinc Oxide - 90:10 |
| 2 | 40,000 ppm | Zinc Borate:Zinc Oxide - 90:10 |
| 3 | 20,000 ppm | Zinc Borate:Zinc Oxide - 50:50 |
| 4 | 40,000 ppm | Zinc Borate:Zinc Oxide - 50:50 |
| 5 | 20,000 ppm | Zinc Borate:Zinc Oxide - 10:90 |
| 6 | 40,000 ppm | Zinc Borate:Zinc Oxide - 10:90 |
| 7 | 0 ppm | Standard Glaze which includes |
| (control) | | some zinc oxide. |

These six samples were tested in accordance with Japanese Industrial. Standard Z 2801:2000, one of the most common test methods for antibacterial efficacy in inorganic ingredients. The organism utilized in the test was E. coli, which is a pathogenic microbe commonly found in human feces, and therefore also commonly found on toilets and other bathroom products. Test results are reported as a percent reduction of bacteria.

TABLE 2

| | | | Relative |
|---|---|---|---|
| | Bacteria on Control | Bacteria on Sample | Reduction |
| Sample | after 24 hours | after 24 hours | of Bacteria |
| 1 | 5.4 × 10⁵ | <1.0 × 10² | >99.98 |
| 2 | 5.4 × 10⁵ | <1.0 × 10² | >99.98 |
| 3 | 5.4 × 10⁵ | <1.0 × 10² | >99.98 |
| 4 | 5.4 × 10⁵ | <1.0 × 10² | >99.98 |
| 5 | 5.4 × 10⁵ | <1.0 × 10² | >99.98 |
| 6 | 5.4 × 10⁵ | <1.0 × 10² | >99.98 |
| 7 | 5.4 × 10⁵ | 5.4 × 10⁵ | 0 |
| (control) | | | |

The above results demonstrate that the glaze according to the invention showed commercially acceptable efficacy against E. Coli relative to the control.

### Example 2

Metal sheet was coated with an enamel glazing composition containing an antimicrobial formulation consisting of 50% zinc oxide and 50% zinc borate (Formulation #1). The antimicrobial formulation was present in the enamel glaze at 2% and 4% (20,000 ppm and 40,000 ppm, respectively) by weight based on the total dry weight of the enamel glaze. The coated samples were dried and fired as described in Example 1 with the exception that the firing temperature was 800° C. in Example 2. Samples for testing were cut from the metal sheet in 1.5 inch squares. The bare metal edges of the tile samples were sealed with paraffin to prevent rusting and contamination from other metal ions.

Efficacy testing was conducted in accordance with Japanese Industrial Standard Z 2801:2000, one of the most common test methods for antimicrobial efficacy with inorganic antimicrobials. For these tests, the method was modified as follows. The inoculum was 0.1 ml of inoculum with approximately 10⁶ Colony Forming Units (CFU)/ml of bacteria, and the 24 hour contact time was conducted at room temperature, as would occur in real use, rather than in an incubator at 37° C.

The test involved inoculating the surface of the sample with 10⁵ organisms with a thin film placed on top of the inoculum to ensure even distribution and intimate contact of the inoculum on the surface of the sample. The organisms used for inoculation were staph aureus (Sa) and Klebsiella pneumoniae (Kp). The inoculum was left in contact with the surface for 24 hours and then recovered into broth. The recovered inoculum was then plated and incubated for 24 hours. The number of surviving colony forming units was then counted using an automated colony counter. The number of surviving organisms was compared to the number in the initial inoculum. The number of surviving organisms for the treated samples was also compared to the number of surviving organisms on the untreated control.

The results are shown in Table 3. The results were analyzed versus the initial inoculum counts (“Organisms Applied”), and the treated samples were compared to the untreated control. Included in the testing was a chromed metal faucet base.

TABLE 3

| | Organisms Applied | | | | % Reduction | | % Reduction | |
|---|---|---|---|---|---|---|---|---|
| | (CFUs) | | 24 Hour Recovery (CFUs) | | versus Inoculum | | versus Control | |
| | Sa | Kp | Sa | Kp | Sa | Kp | Sa | Kp |
| Control | 2.42E+05 | 2.68E+05 | 4.13E+04 | 1.78E+04 | 82.93% | 93.36% | | |
| 2% Form. #1 | 2.48E+05 | 2.54E+05 | 2.20E+03 | <1.00E+02 | 99.11% | 99.96% | 94.67% | 99.44% |
| 4% Form. #1 | 2.39E+05 | 2.66E+05 | <1.00E+02 | 2.50E+03 | 99.96% | 99.06% | 99.76% | 85.96% |
| 2% Form. #1 | 2.43E+05 | 2.69E+05 | <1.00E+02 | <1.00E+02 | 99.96% | 99.96% | 99.76% | 99.44% |
| Metal Faucet | 2.48E+05 | 2.62E+05 | <1.00E+02 | <1.00E+02 | 99.96% | 99.96% | 99.76% | 99.44% |
| Base Piece | | | | | | | | |

All of the samples resulted in significant reductions from the applied inoculum levels. The treated samples and the metal samples had >1 log reductions versus the untreated control (with one exception that is nearly 1 log), and there are >2 log reductions versus the untreated control for the majority of samples. The “1.00E+02” represented the limit of detection for this test. This means that there was actually no growth on the plates for the 24-hour recovery.

### Example 3

Enameled metal samples were prepared as described in Example 2. Samples were treated with the following antimicrobial formulations: a blend ratio of 50/50 of zinc oxide to zinc borate (Formulation #1), a blend ratio of 10/90 zinc oxide to zinc borate (Formulation #2), and a blend ratio of 90/10 zinc oxide to zinc borate (Formulation #3). The formulations were used at 4%, 7% and 10% (40,000 ppm, 70,000 ppm and 100,000 ppm, respectively) by weight based upon the dry weight of the enamel glaze. Three tiles were tested per formulation and concentration. The control was untreated with any antimicrobial formulation. The bare metal edges of the tile samples were sealed with paraffin to prevent rusting and contamination from other metal ions.

Efficacy testing was conducted by the same protocol as described in Example 2. The samples were inoculated with the organism Klebsiella pneumonlae. The initial inoculum level of each sample was 2.04E+05 CFU, determined by plating of the inoculum. The recovered organisms (24 Hour Recovery) for the treated samples were compared to the initial inoculum (% Reduction), and versus the control. The average value from the controls was 5.08E+05 CFU, and this value was used for the comparison versus the control. The “1.00E+02” represented the limit of detection for the test. This means that there was actually no growth on the plates for the 24-hour recovery.

TABLE 4

| | 24 Hour Recovery | | | % Reduction | | | % Reduction vs. Control | | |
|---|---|---|---|---|---|---|---|---|---|
| Sample | A | B | C | A | B | C | A | B | C |
| Control | 4.95E+04 | 1.95E+05 | 1.28E+06 | 75.74% | 4.41% | NR | | | |
| 4% Form. | <1.00E+02 | 2.00E+03 | <1.00E+02 | >99.95% | 99.02% | >99.95% | >99.98% | 99.61% | >99.98% |
| #2 | | | | | | | | | |
| 4% Form. | <1.00E+02 | 5.00E+02 | <1.00E+02 | >99.95% | 99.75% | >99.95% | >99.98% | 99.90% | >99.98% |
| #3 | | | | | | | | | |
| 4% Form. | <1.00E+02 | 1.50E+03 | <1.00E+02 | >99.95% | 99.26% | >99.95% | >99.98% | 99.70% | >99.98% |
| #1 | | | | | | | | | |
| 7% Form. | 3.00E+02 | 9.40E+03 | <1.00E+02 | 99.85% | 95.39% | >99.95% | 99.94% | 98.15% | >99.98% |
| #2 | | | | | | | | | |
| 7% Form. | 5.00E+02 | <1.00E+02 | <1.00E+02 | 99.75% | >99.95% | >99.95% | 99.90% | >99.98% | >99.98% |
| #3 | | | | | | | | | |
| 7% Form. | <1.00E+02 | <1.00E+02 | — | >99.95% | >99.95% | — | >99.98% | >99.98% | — |
| #1 | | | | | | | | | |
| 10% | <1.00E+02 | <1.00E+02 | 3.00E+02 | >99.95% | >99.95% | 99.85% | >99.98% | >99.98% | 99.94% |
| Form. #2 | | | | | | | | | |
| 10% | 5.00E+02 | 1.90E+04 | 1.00E+03 | 99.75% | 90.69% | 99.51% | 99.90% | 96.26% | 99.80% |
| Form. #3 | | | | | | | | | |
| 10% | <1.00E+02 | <1.00E+02 | <1.00E+02 | >99.95% | >99.95% | >99.95% | >99.98% | >99.98% | >99.98% |
| Form. #1 | | | | | | | | | |

As can be seen from the results above, the samples treated with antimicrobial achieved 1 to 3 log reductions versus the average CFUs for the untreated, control samples.

It will therefore be readily understood by those persons skilled in the art that the present invention is susceptible of broad utility and application. Many embodiments and adaptations of the present invention other than those herein described, as well as many variations, modifications and equivalent arrangements, will be apparent from or reasonably suggested by the present invention and the foregoing description thereof, without departing from the substance or scope of the present invention. Accordingly, while the present invention has been described herein in detail in relation to its preferred embodiment, it is to be understood that this disclosure is only illustrative and exemplary of the present invention and is made merely for purposes of providing a full and enabling disclosure of the invention. The foregoing disclosure is not intended or to be construed to limit the present invention or otherwise to exclude any such other embodiments, adaptations, variations, modifications and equivalent arrangements.

1. An antimicrobial enamel glazing composition comprising a quantity of zinc borate sufficient to achieve a commercially acceptable level of antimicrobial efficacy.

2. The antimicrobial enamel glazing composition according to claim 1, wherein the antimicrobial ceramic glazing composition exhibits at least a 90% relative reduction of bacteria after 24 hours as compared to an untreated control when tested in accordance with Japanese Industrial Standard Z 2801:2000.

3. The antimicrobial enamel glazing composition according to claim 1, wherein the antimicrobial ceramic glazing composition has a concentration of zinc borate of at least about 5,000 ppm.

4. The antimicrobial enamel glazing composition according to claim 3, wherein the antimicrobial ceramic glazing composition has a concentration of zinc borate of about 20,000 ppm to about 40,000 ppm.

5. The antimicrobial enamel glazing composition according to claim 1, further comprising a quantity of zinc oxide.

6. The antimicrobial enamel glazing composition according to claim 5, wherein the antimicrobial ceramic glazing composition has a ratio of zinc borate to zinc oxide from about 10:90 to about 90:10.

7. The antimicrobial enamel glazing composition according to claim 5, wherein the antimicrobial ceramic glazing composition has a combined concentration of zinc borate and zinc oxide of at least about 5,000 ppm.

8. The antimicrobial enamel glazing composition according to claim 7, wherein the antimicrobial ceramic glazing composition has a combined concentration of zinc borate and zinc oxide of about 20,000 ppm to about 40,000 ppm.

9. An enameled article comprising the antimicrobial enamel glazing composition according to claim 1.

10. An enameled article comprising the antimicrobial enamel glazing composition according to claim 5.

11. The enameled article according to claim 9, wherein the enameled article is selected from the group consisting of sinks, washbasins, bathtubs, eating utensils, pots, pans, jewelry, decorative items, towel rails, soap holders, toilet roll holders, water control fixtures, and tiles.

12. The enameled article according to claim 10, wherein the enameled article is selected from the group consisting of sinks, washbasins, bathtubs, eating utensils, pots, pans, jewelry, decorative items, towel rails, soap holders, toilet roll holders, water control fixtures, and tiles.

13. An enameled article that exhibits antimicrobial properties, said article comprising a metal substrate having at least one surface and a glaze on a portion of said surface, said glaze comprising a quantity of zinc borate sufficient to achieve a commercially acceptable level of antimicrobial efficacy.

14. The enameled article according to claim 13, wherein the enameled article exhibits at least a 90% relative reduction of bacteria after 24 hours as compared to an untreated control when tested in accordance with Japanese Industrial Standard JIS Z 2801:2000.

15. The enameled article according to claim 13, wherein the glaze has a concentration of zinc borate of at least about 5,000 ppm.

16. The enameled article according to claim 15, wherein the glaze has a concentration of zinc borate of about 20,000 ppm to about 40,000 ppm.

17. The enameled article according to claim 13, further comprising a quantity of zinc oxide.

18. The enameled article according to claim 17, wherein the glaze has a ratio of zinc borate to zinc oxide of from about 10:90 to about 90:10.

19. The enameled article according to claim 17, wherein the glaze has a combined concentration of zinc borate and zinc oxide of at least about 5,000 ppm.

20. The enameled article according to claim 19, wherein the glaze has a combined concentration of zinc borate and zinc oxide of about 20,000 ppm to about 40,000 ppm.

21. The enameled article according to claim 13, wherein the enameled article is selected from the group consisting of sinks, washbasins, bathtubs, eating utensils, pots, pans, jewelry, decorative items, towel rails, soap holders, toilet roll holders, water control fixtures, and tiles.

22. The enameled article according to claim 17, wherein the enameled article is selected from the group consisting of sinks, washbasins, bathtubs, eating utensils, pots, pans, jewelry, decorative items, towel rails, soap holders, toilet roll holders, water control fixtures, and tiles.

23. A method for forming an enamel glaze having antimicrobial properties, the method comprising:
preparing an enamel glazing composition, and
adding to the enamel glazing composition a quantity of zinc borate sufficient to achieve a commercially acceptable level of antimicrobial efficacy.

24. The method according to claim 23, wherein the enamel glazing composition has a concentration of zinc borate of at least about 5,000 ppm.

25. The method according to claim 24, wherein the enamel glazing composition has a concentration of zinc borate of about 20,000 ppm to about 40,000 ppm.

26. The method according to claim 23, further comprising adding a quantity of zinc oxide to the enamel glazing composition.

27. The method according to claim 26, wherein the enamel glazing composition has a ratio of zinc borate to zinc oxide of from about 10:90 to about 90:10.

28. The method according to claim 26, wherein the enamel glazing composition has a combined concentration of zinc borate and zinc oxide of at least about 5,000 ppm.

29. The method according to claim 28, wherein the enamel glazing composition has a combined concentration of zinc borate and zinc oxide of about 20,000 ppm to about 40,000 ppm.

30. The method according to claim 23, further comprising coating a substrate with the antimicrobial enamel glazing composition.

31. The method according to claim 26, further comprising coating a substrate with the antimicrobial ceramic glazing composition.

32. The method according the claim 30, further comprising firing the coated substrate.

33. The method according the claim 31, further comprising firing the coated substrate.

34. The method according to claim 32, wherein the fired coated substrate exhibits at least a 90% relative reduction of bacteria after 24 hours as compared to an untreated control when tested in accordance with Japanese Industrial Standard Z 2801:2000.

35. The method according to claim 33, wherein the fired coated substrate exhibits at least a 90% relative reduction of bacteria after 24 hours as compared to an untreated control when tested in accordance with Japanese Industrial Standard Z 2801:2000.

36. The method according to claim 32, wherein the fired coated substrate is selected from the group consisting of sinks, washbasins, bathtubs, eating utensils, pots, pans, jewelry, decorative items, towel rails, soap holders, toilet roll holders, water control fixtures, and tiles.

37. The method according to claim 33, wherein the fired coated substrate is selected from the group consisting of sinks, washbasins, bathtubs, eating utensils, pots, pans, jewelry, decorative items, towel rails, soap holders, toilet roll holders, water control fixtures, and tiles.
